(19) United States
(12) Patent Application Publication
(10) Pub. No.: US 2008/0242856 A1
(43) Pub. Date: Oct. 2, 2008

(54) Extracts of taiwanofungus camphoratus with a capacity for inhibiting the activity of matrix metalloproteinases and method for preparing the same

(75) Inventor: Ming-Yong Lue, Taichung City (TW)

Correspondence Address: CHARLES E. BAXLEY, ESQUIRE, 90 JOHN STREET, SUITE 309, NEW YORK, NY, 10038, US

(73) Assignee: Funci Biotechnology Co., Ltd.

(21) Appl. No.: 11/732,190

(22) Filed: Apr. 2, 2007

(51) Int. Cl. C07H 1/08

(52) U.S. Cl. 536/127

(57) ABSTRACT

Provided is a method for preparing the extracts of Taiwanofungus camphoratus with a capacity for inhibiting the activity of matrix metalloproteinases. This product is obtained from submerged culture for mycelium of the isolated strain of Taiwanofungus camphoratus. Various concentrations of ethanol precipitation with the cultural medium comprising exopolysaccharides have been performed to separate several fractions of polysaccharides with different molecular weights. Especially, this novel mixture containing exopolysaccharides with molecular weights between 1000 and 30000 exhibits the suppressing ability on the activity of matrix metalloproteinases (MMPs). In addition, also provided is an entire process for preparing this product including the procedures for agar plate culture, mycelium culture, and extraction by means of various concentrations of ethanol.

### BACKGROUND OF THE INVENTION

1. Technical Field

The present invention relates to extracts of Taiwanofungus camphoratus with a capacity for inhibiting the activity of matrix metalloproteinases and the method for preparing the same. Various concentrations of ethanol precipitation with the cultural medium comprising exopolysaccharides have been performed to obtain this novel mixture containing exopolysaccharides exhibits the suppressing ability on the activity of matrix metalloproteinases (MMPs).

2. Description of Related Art

Taiwanofungus camphoratus is an endemic species of Taiwan and is known only occurring on trunk of Cinnamomum kanehirai Hay, which is a native conservation tree species of Taiwan. According to previous reports, the active principle of Taiwanofungus camphorates comprises polysaccharides and triterpenoids.

Presently, extracts of Taiwanofungus camphorates have been broadly applied to immune regulation, stimulating hematopoiesis, enhancing human exercise ability, antifatigue, and medical usage such as the treatments for cancers or tumor diseases and the treatment for Hepatitis B.

Further, the ethanolic extract of Taiwanofungus camphoratus mycelium also functions as a sweeper of superoxide anions and hydroxyl free radicals for its anti-oxidant capability. Besides, Polysaccharide in the fruiting bodies and mycelium of Taiwanofungus camphoratus is able to facilitate remedying liver injuries induced by carbon tetrachloride.

However, extracts of Taiwanofungus camphoratus Polysaccharide used in the above-mentioned applications are typically derived through large-scale ethanol precipitation by implementing ethanol with a concentration ranging from 75.0% to 95.0%. That is to say, total polysaccharide is the prime extract used in the previous discussed applications.

### SUMMARY OF THE INVENTION

The present invention discloses an extract of Taiwanofungus camphoratus with a capacity for inhibiting the activity of matrix metalloproteinases and the method of preparing the same. This product is obtained from submerged culture for mycelium of the isolated strain of Taiwanofungus camphoratus. Various concentrations of ethanol precipitation with the cultural medium comprising exopolysaccharides have been performed to separate ten fractions of polysaccharides with different molecular weights. Then a gelatin-based zymography of matrix metalloproteinases using gelatin as substrate has been conducted. And it is found that the polysaccharides derived from ethanol precipitation done with ethanol having concentrations respectively ranging from 50.0% to 66.7% and ranging from 66.7% to 75.0% with the molecular weights ranging between 1000 and 30000 exhibit the suppressing ability on the activity of matrix metalloproteinase-2 (MMP-2). Thus, the present invention relates to efficiently applying the exopolysaccharides with molecular-weights between 1000 and 30000 derived through ethanol precipitation of Taiwanofungus camphoratus using ethanol having concentrations ranging from 50.0% to 75.0% (“ethanol concentration 50.0% to 66.7%” and “ethanol concentration 66.7% to 75.0%”) to the suppression of the activity of matrix metalloproteinase-2.

### BRIEF DESCRIPTION OF THE DRAWINGS

The invention as well as a preferred mode of use, further objectives and advantages thereof, will best be understood by reference to the following detailed description of an illustrative embodiment when read in conjunction with the accompanying drawings, wherein:

FIG. 1 is a list containing the various concentrations of ethanol precipitation with the cultural medium comprising exopolysaccharides for separating 10 fractions of polysaccharides;

FIG. 2 describes the method of extracting the 10 fractions of the exopolysaccharides obtained from submerged culture for mycelium of the isolated strain of Taiwanofungus camphoratus;

FIG. 3 is a table listing the retention time and molecular weight distribution of the 10 fractions of the exopolysaccharides obtained from submerged culture for mycelium of the isolated strain of Taiwanofungus camphoratus evaluated by gel permeation chromatography; and

FIG. 4 illustrates the resultant activity of matrix metalloproteinases in the culture solution of 3T3 culture media after treating with the groups of fractions 1˜5 and fractions 6˜10 of the exopolysaccharides obtained from submerged culture for mycelium of the isolated strain of Taiwanofungus camphoratus respectively wherein the dosage of exopolysaccharides is 0.5 mg/ml and the treating duration is 48 hours respectively and it can be learned from FIG. 4 that the Taiwanofungus camphoratus exopolysaccharide of fraction 5 (lane 5) (obtained by ethanol precipitation with 50.0% to 66.7% ethanol) and the Taiwanofungus camphoratus exopolysaccharide of fraction 6 (lane 6) (obtained by ethanol precipitation with 66.7% to 75.0% ethanol) exhibit the outstanding suppressing ability on the activity of matrix metalloproteinase-2.

### DETAILED DESCRIPTION OF THE PREFERRED EMBODIMENT

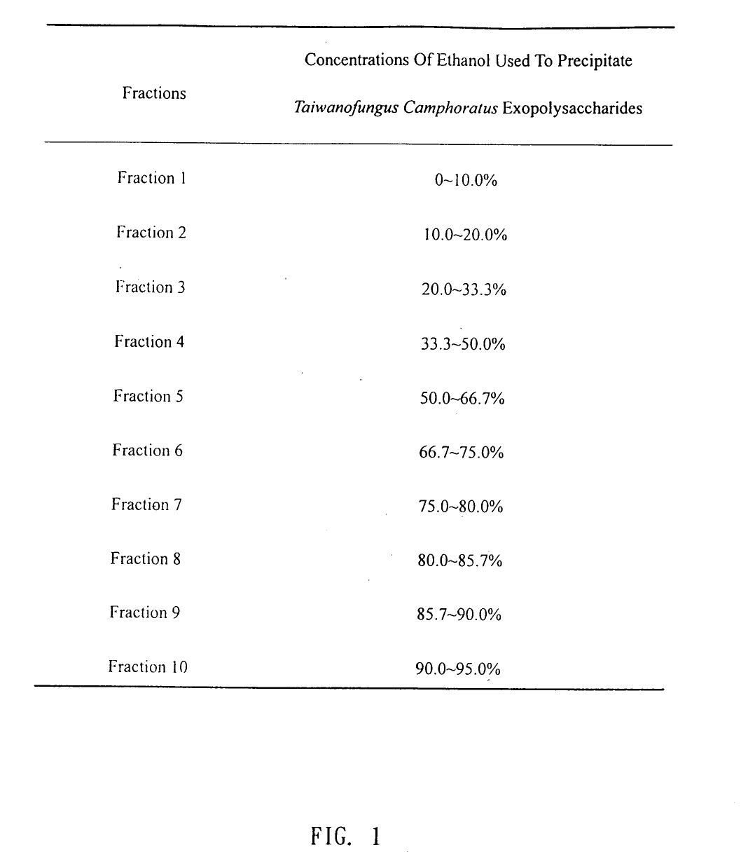

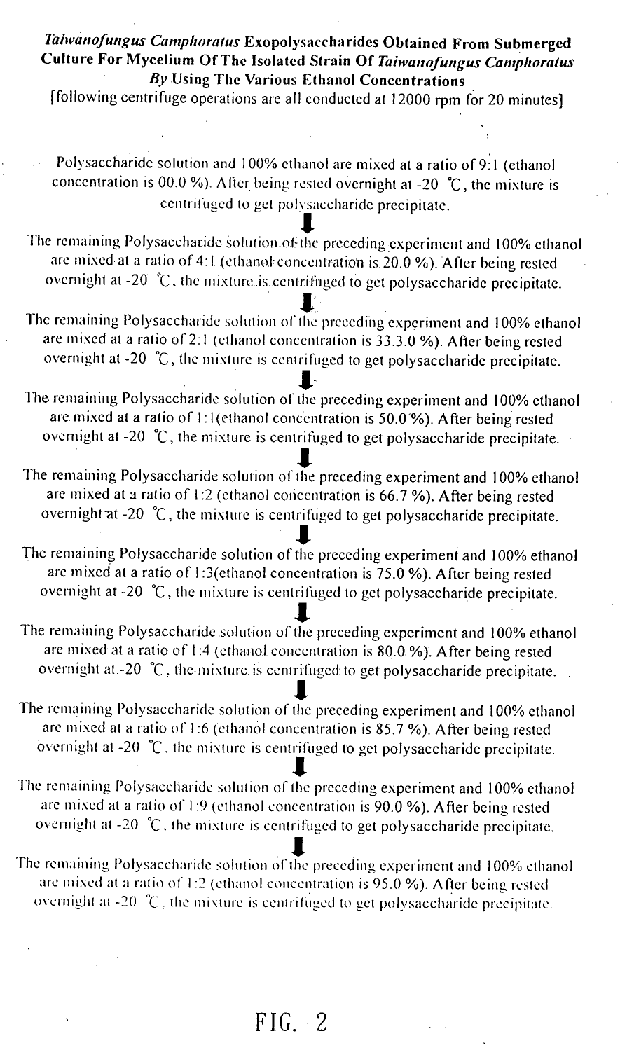

The method for preparing the extracts of Taiwanofungus camphoratus with a capacity for inhibiting the activity of matrix metalloproteinases, referring to FIG. 1, implements 10 concentration ranges of ethanol precipitation which are respective 0˜10.0%, 10.0˜20.0%, 20.0˜33.3%, 33.3˜50.0%, 50.0˜66.7%, 66.7˜75.0%, 75.0˜80.0%, 80.0˜85.7%, 85.7˜90.0% and 90.0˜95.0%. The ethanol precipitation for separating the exopolysaccharides obtained from submerged culture for mycelium of the isolated strain of Taiwanofungus camphoratus by implementing the stepped ethanol concentrations (from lower ethanol concentrations to higher ethanol concentrations) primarily uses the various ethanol concentrations to derive 10 fractions of the exopolysaccharides obtained from submerged culture for mycelium of the isolated strain of Taiwanofungus camphoratus. The applied ethanol precipitation is as shown in FIG. 2 and will be described briefly as hereinafter.

(1) Polysaccharide solution and 100% ethanol are mixed at a ratio of 9:1 (ethanol concentration is 10.0%) and after rested overnight at −20° C., the mixture is centrifuged (at 12000 rpm for 20 minutes) to get polysaccharide precipitate.

(2) The remaining Polysaccharide solution of the preceding experiment and 100% ethanol are mixed at a ratio of 4:1 (ethanol concentration is 20.0%). After being rested overnight at −20° C., the mixture is centrifuged (at 12000 rpm for 20 minutes) to get polysaccharide precipitate.

(3) The remaining Polysaccharide solution of the preceding experiment and 100% ethanol are mixed at a ratio of 2:1 (ethanol concentration is 33.3%). After being rested overnight at −20° C., the mixture is centrifuged (at 12000 rpm for 20 minutes) to get polysaccharide precipitate.

(4) The remaining Polysaccharide solution of the preceding experiment and 100% ethanol are mixed at a ratio of 1:1 (ethanol concentration is 50.0%). After being rested overnight at −20° C., the mixture is centrifuged (at 12000 rpm for 20 minutes) to get polysaccharide precipitate.

(5) The remaining Polysaccharide solution of the preceding experiment and 100% ethanol are mixed at a ratio of 1:2 (ethanol concentration is 66.7%). After being rested overnight at −20° C., the mixture is centrifuged (at 12000 rpm for 20 minutes) to get polysaccharide precipitate.

(6) The remaining Polysaccharide solution of the preceding experiment and 100% ethanol are mixed at a ratio of 1:3 (ethanol concentration is 75.0%). After being rested overnight at −20° C., the mixture is centrifuged (at 12000 rpm for 20 minutes) to get polysaccharide precipitate.

(7) The remaining Polysaccharide solution of the preceding experiment and 100% ethanol are mixed at a ratio of 1:4 (ethanol concentration is 80.0%). After being rested overnight at −20° C., the mixture is centrifuged (at 12000 rpm for 20 minutes) to get polysaccharide precipitate.

(8) The remaining Polysaccharide solution of the preceding experiment and 100% ethanol are mixed at a ratio of 1:6 (ethanol concentration is 85.7%). After being rested overnight at −20° C., the mixture is centrifuged (at 12000 rpm for 20 minutes) to get polysaccharide precipitate.

(9) The remaining Polysaccharide solution of the preceding experiment and 100% ethanol are mixed at a ratio of 1:9 (ethanol concentration is 90.0%). After being rested overnight at −20° C., the mixture is centrifuged (at 12000 rpm for 20 minutes) to get polysaccharide precipitate.

(10) The remaining Polysaccharide solution of the preceding experiment and 100% ethanol are mixed at a ratio of 1:19 (ethanol concentration is 95.0%). After being rested overnight at −20° C., the mixture is centrifuged (at 12000 rpm for 20 minutes) to get polysaccharide precipitate.

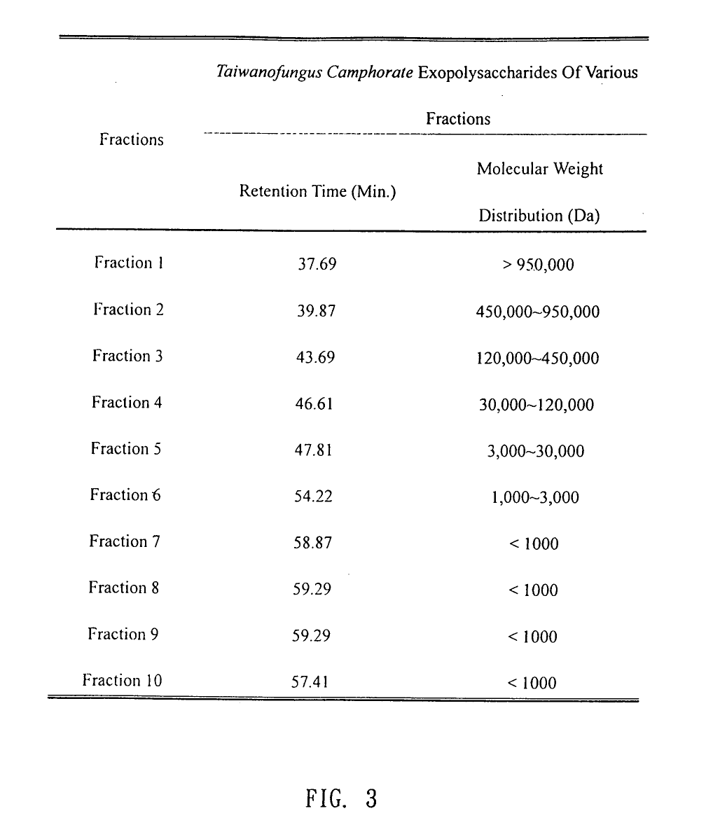

By implementing various ethanol concentrations (the stepped ethanol concentrations) to precipitate Taiwanofungus camphoratus exopolysaccharides, 10 fractions with different molecular weights of polysaccharide mixtures can be derived. The retention time and molecular weight distribution of the 10 fractions of the exopolysaccharides evaluated by gel permeation chromatography can be seen in FIG. 3. Then T3T mouse fibroblast culture solution subcultured for 48 hours is collected (containing matrix metalloproteinases (MMPs)) and put to react with 0.5 mg/ml of each of the 10 fractions of the exopolysaccharides culture solution respectively for 0 or 48 hours. Then a gelatin-based zymography of matrix metalloproteinases using gelatin as substrate is conducted to detect whether the Taiwanofungus camphoratus exopolysaccharides with various molecular weights have suppressing ability on the enzyme activity of MMPs (MMp-9 and MMP-2). The “culture solution” used in the above-discussed experiment is the culture solution that has been implemented to cultivate T3T cells for 48 hours and is separated from the T3T cells therein. Thus, only the “culture solution” is used in the experiment.

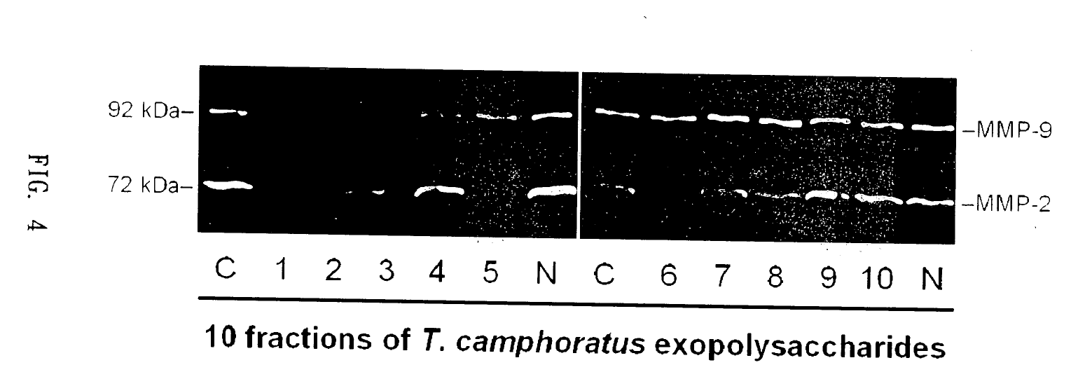

By means of gelatin-based zymography, it is proved that the fraction 5 (Taiwanofungus camphoratus exopolysaccharide obtained from ethanol precipitation with 50.0% to 66.7% ethanol) and the fraction 6 (Taiwanofungus camphoratus exopolysaccharide obtained from ethanol precipitation with 66.7% to 75.0% ethanol) exhibit the outstanding suppressing ability on the activity of matrix metalloproteinase-2. Therefore, the present invention applies the Taiwanofungus camphoratus exopolysaccharides precipitated from the Taiwanofungus camphoratus exopolysaccharide mixtures of the fraction 5 and fraction 6, i.e. the exopolysaccharide mixtures with 50.0% to 75.0% ethanol concentrations (“ethanol concentration 50.0% to 66.7%” and “ethanol concentration 66.7% to 75.0%”) to perform the optimum suppression of the activity of matrix metalloproteinase-2, as shown in FIG. 4. Wherein the molecular weight distribution of the Taiwanofungus camphoratus exopolysaccharides of the fraction 5 and fraction 6 is ranging from 1000 to 30000, as shown in FIG. 3.

The disclosed method for preparing the extracts of Taiwanofungus camphoratus with a capacity for inhibiting the activity of matrix metalloproteinases which implements various concentrations of ethanol precipitation with Taiwanofungus camphoratus mycelium cultural medium to exact exopolysaccharides is describe in detail hereinafter. The suspension of submerged culture for mycelium of the isolated strain of Taiwanofungus camphoratus is centrifuged at 8000 rpm for being separated into solid mycelium and clear culture solution and then the clear culture solution is mixed with 100% ethanol at a ratio of 1:1 (ethanol concentration is 50.0%). After being rested overnight at −20° C., the mixture is further centrifuged (at 12000 rpm for 20 minutes) to get exopolysaccharide precipitate with larger molecular weight (molecular weight larger than 3000). The centrifuged clear solution (containing 50.0% ethanol) is mixed with 100% ethanol in proportion for raising the ethanol concentration to 75.0%. After being rested overnight at −20° C., the mixture is further centrifuged (at 12000 rpm for 20 minutes) to get exopolysaccharide precipitate with molecular weights between 1000 and 30000. Please refer to FIGS. 5 and 6, the Taiwanofungus camphoratus exopolysaccharide obtained through these steps exhibits the suppressing ability on the activity of MMP-2.

The disclosed method for preparing the extracts of Taiwanofungus camphoratus with a capacity for inhibiting the activity of matrix metalloproteinases includes the procedures for agar plate culture, mycelium culture, and extraction by means of various concentrations of ethanol. The preparing procedures comprises:

(1) Agar plate culture of the isolated strain of Taiwanofungus camphoratus: Taiwanofungus camphoratus mycelium is inoculated in a round flat petri dish and cultured in the culture media containing of 2% malt extract, 2% glucose, 0.1% bacto peptone and 2% bacto agar in a 26° C. incubator for 20 to 30 days.

(2) Submerged culture for mycelium of the isolated strain of Taiwanofungus camphoratus: The mycelium in the round flat petri dish is taken to be inoculated in a flask and cultured in the starch-free medium containing 2% glucose, 2% malt extract, 1% bacto peptone, 0.05% MgSO₄, 0.05% KH₂PO₄, 0.05% K₂HPO₄ in an orbital shaking incubator operated at 105 rpm, 28° C., pH 5.5 (before inoculation) for one week.

(3) Submerged culture for mycelium of the isolated strain of Taiwanofungus camphoratus in a 5-liter fermentation tank: The culture of the submerged culture in the flask is taken to be inoculated in a fermentation tank and cultured in the medium having the same formula of the medium used previously at 120 rpm, 28° C., and aeration rate of 1 vvm for about 5 to 7 days.

(4) Refinement of extract of Taiwanofungus camphoratus (exopolysaccharides with molecular weights between 1000 and 30000): The suspension of submerged culture for mycelium of the isolated strain of Taiwanofungus camphoratus is centrifuged at 8000 rpm for being separated into solid mycelium and clear culture solution and then the clear culture solution is mixed with 100% ethanol at a ratio of 1:1 (ethanol concentration is 50.0%). After being rested overnight at −20° C., the mixture is further centrifuged (at 12000 rpm for 20 minutes) to get exopolysaccharide precipitate with larger molecular weights (molecular weights larger than 3000). The centrifuged clear solution (containing 50.0% ethanol) is mixed with 100% ethanol in proportion for raising the ethanol concentration to 75.0%. After being rested overnight at −20° C., the mixture is further centrifuged (at 12000 rpm for 20 minutes) to get exopolysaccharide precipitate with molecular weights between 1000 and 30000.

To sum up, the disclosed method for preparing the extracts of Taiwanofungus camphoratus with a capacity for inhibiting the activity of matrix metalloproteinases implements various concentrations of ethanol precipitation with Taiwanofungus camphoratus mycelium cultural medium to extract exopolysaccharides that exhibit the suppressing ability on the activity of matrix metalloproteinases-2 (MMP-2). The molecular weight of the exopolysaccharides ranges from 1000 to 30000 and the concentration of the ethanol used to refine Taiwanofungus camphoratus exopolysaccharides ranges from 50.0% to 75.0%.

Although a particular embodiment of the invention has been described in detail for purposes of illustration, it will be understood by one of ordinary skill in the art that numerous variations will be possible to the disclosed embodiments without going outside the scope of the invention as disclosed in the claims.

What is claimed is:

1. A method for preparing the extracts of Taiwanofungus camphoratus with a capacity for inhibiting the activity of matrix metalloproteinases, wherein the extract of Taiwanofungus camphoratus obtained from submerged culture for mycelium of the isolated strain of Taiwanofungus camphoratus is a unique polysaccharide to Taiwanofungus camphoratus and has molecular weights between 1000 and 30000.

2. The method of claim 1, wherein the extract of Taiwanofungus camphoratus is obtained from the suspension which is a result of the submerged culture for mycelium of the isolated strain of Taiwanofungus camphoratus.

3. The method of claim 2, wherein the extract of Taiwanofungus camphoratus is obtained from the clear culture solution of the suspension which is a result of the submerged culture for mycelium of the isolated strain of Taiwanofungus camphoratus.

4. The method of claim 3, wherein the extract of Taiwanofungus camphoratus is obtained from the exopolysaccharides contained in the clear culture solution of the suspension which is a result of the submerged culture for mycelium of the isolated strain of Taiwanofungus camphoratus.

5. The method of claim 4, wherein the extract of Taiwanofungus camphoratus is obtained by refining the exopolysaccharides contained in the clear culture solution of the suspension which is a result of the submerged culture for mycelium of the isolated strain of Taiwanofungus camphoratus.

6. The method of claim 5, wherein the concentration of the ethanol used to refine Taiwanofungus camphoratus exopolysaccharides ranges form 50.0% to 75.0%.

7. A method for preparing the extracts of Taiwanofungus camphoratus with a capacity for inhibiting the activity of matrix metalloproteinases, wherein the extract of Taiwanofungus camphoratus obtained from submerged culture for mycelium of the isolated strain of Taiwanofungus camphoratus is a unique polysaccharide to Taiwanofungus camphoratus and has molecular weights between 1000 and 30000, comprising following procedures:
(1) Agar plate culture of the isolated strain of Taiwanofungus camphoratus: inoculating Taiwanofungus camphoratus mycelium in a round flat petri dish for being cultured in the culture media containing of 2% malt extract, 2% glucose, 0.1% bacto peptone and 2% bacto agar in a 26° C. incubator for 20 to 30 days;
(2) Submerged culture for mycelium of the isolated strain of Taiwanofungus camphoratus: inoculating the mycelium in the round flat petri dish into a flask for being cultured in the starch-free medium containing 2% glucose, 2% malt extract, 1% bacto peptone, 0.05% MgSO₄, 0.05% KH₂PO₄, 0.05% K₂HPO₄ in an orbital shaking incubator operated at 105 rpm, 28° C., pH 5.5 (before inoculation) for one week;
(3) Submerged culture for mycelium of the isolated strain of Taiwanofungus camphoratus in a 5-liter fermentation tank: inoculating the culture of the submerged culture in the flask into a fermentation tank for being cultured in the medium having the same formula of the medium used previously at 120 rpm, 28° C., and aeration rate of 1 vvm for about 5 to 7 days; and
(4) Refinement of extract of Taiwanofungus camphoratus (exopolysaccharides with molecular weights between 1000 and 30000): centrifuging the suspension of submerged culture for mycelium of the isolated strain of Taiwanofungus camphoratus at 8000 rpm for separating it into solid mycelium and clear culture solution; mixing the clear culture solution with 100% ethanol at a ratio of 1:1 (ethanol concentration is 50.0%); resting the mixture overnight at −20° C.; centrifuging the mixture (at 12000 rpm for 20 minutes) to get Taiwanofungus camphoratus exopolysaccharide precipitate with larger molecular weights (molecular weights larger than 3000); mixing the centrifuged clear solution (containing 50.0% ethanol) with 100% ethanol in proportion for raising the ethanol concentration to 75.0%; resting the mixture overnight at −20° C.; and centrifuging the mixture (at 12000 rpm for 20 minutes) to get Taiwanofungus camphoratus exopolysaccharide precipitate with molecular weights between 1000 and 30000.
